(19) United States
(12) Patent Application Publication
(10) Pub. No.: US 2014/0018552 A1
(43) Pub. Date: Jan. 16, 2014

(54) USE OF COMPOUNDS ISOLATED FROM MORUS BARK

(71) Applicants: Hwan Bong Chang, Gyeonggi-do (KR); Joobyoung Yoon, Gyeonggi-do (KR); Hyun Yong Lee, Gyeonggi-do (KR); Hyun Sik Choi, Gyeonggi-do (KR); Hyung Bok Lee, Gyeonggi-do (KR)

(72) Inventors: Hwan Bong Chang, Gyeonggi-do (KR); Joobyoung Yoon, Gyeonggi-do (KR); Hyun Yong Lee, Gyeonggi-do (KR); Hyun Sik Choi, Gyeonggi-do (KR); Hyung Bok Lee, Gyeonggi-do (KR)

(73) Assignee: Dong Wha Pharm. Co., Ltd., Seoul (KR)

(21) Appl. No.: 14/007,603

(22) PCT Filed: Mar. 26, 2012
(86) PCT No.: PCT/KR12/02162

§ 371 Date: Sep. 25, 2013

(30) Foreign Application Priority Data
Mar. 28, 2011 (KR) 10-2011-0027789

(51) Int. Cl. A61K 31/352; A23L 1/30; A61K 31/05

(52) U.S. Cl. CPC A61K 31/352; A61K 31/05; A23L 1/30
USPC 549/382; 549/403; 549/381; 568/729; 549/387

(57) ABSTRACT

The present invention provides an AGE inhibitor and a health functional food for inhibiting an occurrence of diabetic complications. Particularly, the present invention comprises a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Morus Bark as an active ingredient. The compounds disclosed above inhibit the production of AGE which is a causative substance of diabetic complications. Thus, the compounds could be used as an AGE inhibitor and a health functional food for inhibiting diabetic nephropathy, diabetic retinopathy and diabetic neuropathy.

### BACKGROUND OF THE INVENTION

The present invention relates to advanced glycation end product (AGE) inhibitors and health functional foods for inhibiting an occurrence of diabetic complications, comprising a compound isolated from Morus Bark as active ingredients.

Diabetes, one of the typical adult diseases, is known for lasting hyperglycemia by abnormal secretion or function of insulin all over the world. Recently, a prevalence rate of diabetes in Korea has been rapidly increased by the westernization of dietary life and aging population growth.

Diabetic complications are divided into two groups, acute diseases and chronic diseases. The acute diseases include diabetic ketoacidosis, hyperglycemic hyperosmolar syndrome and so on. The chronic diseases include microvascular diseases (such as diabetic nephropathy, diabetic retinopathy and diabetic neuropathy) and macrovascular diseases (such as diabetic cardiomyopathy and cerebrovascular diseases).

Drugs for preventing and treating the diseases induced by the said diabetic complications are advanced glycation end product inhibitors, aldose reductase inhibitors, transforming growth factor-β receptor inhibitors and so on.

An advanced glycation end product inhibitor suppresses an occurrence of diabetic complications resulted from advanced glycation end products (AGEs) produced by nonenzymatic glycation of proteins due to the continuation of hyperglycemia. It also inhibits the production of AGEs.

Hyperglycemia being lasted, the structural and functional changes of protein and fat are brought about by the nonenzymatic combination or rearrangement of protein, fat, and reducing sugar including glucose in the blood. During this process, the irreversible AGEs are produced. Moreover, AGEs produced by hyperglycemia are known for inducing diabetic complications by signal transmit through the binding to receptor for AGEs (RAGE) on the cell surface and the cross-linking with extracellular matrix protein such as collagen or fibronectin. (Schmidt, A. M., et al., 2000. Trends Endocrinol. Metab. 11, 368-375).

Specifically, AGEs induce diabetic nephropathy by activating Smad-2/3 in TGF-β-dependent or TGF-β-independent way (Li, J. H., et el., 2004, FASEB J. 18, 176-178; Fukami K., et al., 2004, Kidney Int. 66, 2137-2147; Chung, C. Kt., et al., 2010, J. Am. Soc. Nephrol., 21, 249-260). Also, it is reported that AGEs induce diabetic retinopathy and diabetic neuropathy by the interaction with a receptor for advanced glycation end product (RAGE). (Barile G. R. et al., 2005, Invest Ophthalmol Vis Sci. 46(8), 2916-2924; Toth C., et al., 2008, Diabetes. 57(4), 1002-1017)

In the experiment with animals to which AGE inhibitor is administered, the occurrence of diabetic nephropathy (Osicka T. T. et al., 2000, Diabetes. 49(1), 87-93; Yang C. W., et al., 1994, Proc Natl Acad Sci USA. 91(20) 9436-9440), diabetic retinopathy (Hammes H. P., et al., 1991, Proc Natl Acad Sci USA. 88(24), 11555-11558) and diabetic neuropathy (Duran-Jimenez B., et al., 2009, Diabetes. 58(12), 2893-2903) were significantly inhibited.

Among typical AGE inhibitors, there are aminoguanidine an pyridoxamine (product: pyridoline). However, the development of aminoguanidine was stopped because of the toxicity related to vitamin B deficiency in the clinical trial (phase 3). Also, the clinical trial (phase 3) of pyridoxamine is in the course of preparation after completing the trial (phase 2) recently so there is no drug commercialized yet

Meanwhile, Morus Bark, the herb made of a root bark in Morus, is known to have pharmacological effects such as antitussive (cough-suppressing), diuretic, hypotensive (blood pressure reducing), sedative, analgesic, antipyretic (fever-reducing), antispasmodic, and antibacterial actions. (Doosan Encyclopedia, Encyber & Encyber.com)

A lot of components were isolated from Morus Bark and there have been many studies about them. Moracin O, moracin P and mulberrofuran H isolated from Morus Bark are disclosed to have an effect on inhibiting the activity of HF-1 (Hypoxia Inducible Factor-1) which induces various cancers and diabetic retinopathy. (Korean Patent Application No. 2007-78888) Also, moracin-M is reported to have an effect on reducing blood sugar in rat having diabetes. (Zang M., et al., 2009, Fitoterapia 80(8) 475-477) Kuwanon-L has an effect on inhibiting PTP1B1 (Protein tyrosin phosphatase 1B1). (Cui L. et al., 2006, Bioorg. Med. Chem. Lett. 16(5) 1426-1429) Morin 3,5,7,2′,4′-pentahydroxyflavone is reported its antagonism of TGF-receptor II and its effect on inhibiting diastatic action of low density lipoprotein. (Gaffari M. A., et al., 2007, Iran Biomed. J. 11(3) 185-191; Shimanuki T., et al., 2007, Oncogene 26(23) 3311-3320)

In addition to the aforementioned, mulberrofuran G, mulberrofuran K, kuwanon G, kuwanon Z, oxyresveratrol, 2′,4′,5,7-tetrahydroxyflavanone, morusignin L and dihydromorin are also known substances isolated from Morus Bark.

The compounds of mulberrofuran G, mulberrofuran K and kuwanon G isolated from Morus Bark are Diels-Alder type adducts and kuwanon Z is Silbene derivative. Also, oxyresveratrol is Coumarin derivative. 2′,4′,5,7-tetrahydroxyflavanone, morusignin L and dihydromorin are Flavonoid derivatives. The uses are as follows:

Mulberrofuran G as represented in Formula 1 has an effect on hindering tyrosinase (Zheng Z. P., et al., 2010, Agric. Food Chem. 58(9) 5368-5373) and has antioxidative activities (Dai S. J., et al., 2004, Chem. Pharm. Bull (Tokyo) 52(10) 1190-1193).

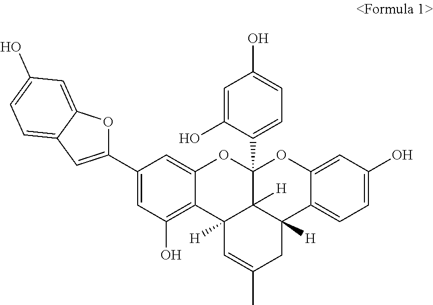

Mulberrofuran K as represented in Formula 2 is known as its antioxidative effect (Dai S. J., et al., 2004, Chem. Pharm. Bull (Tokyo) 52(10) 1190-1193).

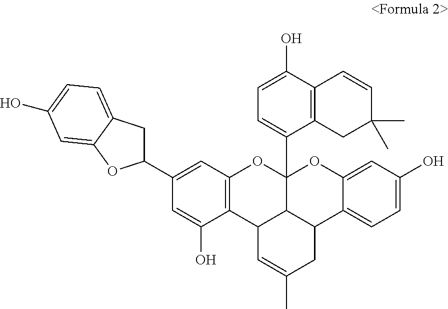 [SMILES: CC1=CC2c3c(O)cc(C4Cc5ccc(O)cc5O4)cc3OC3(c4ccc(O)c5c4CC(C)(C)C=C5)Oc4cc(O)ccc4C(C1)C23]

Kuwanon G as represented in Formula 3 has antibacterial activities (Park. K. M., et al., 2003, J. Ethnopharmacol. 84(2-3) 181-185) and antagonism with bombesin receptor (Mihara S., et al., 1995, Biochem Biophys Res Commun. 15; 213(2):594-9).

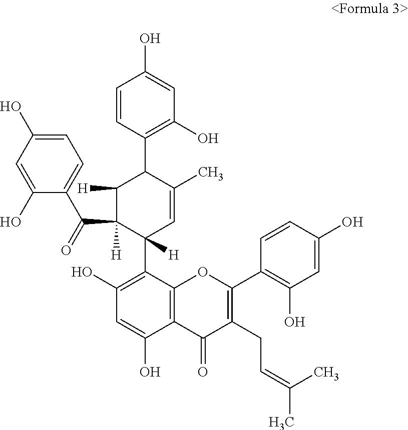

The activity of kuwanon Z as represented in Formula 4 has not been studied.

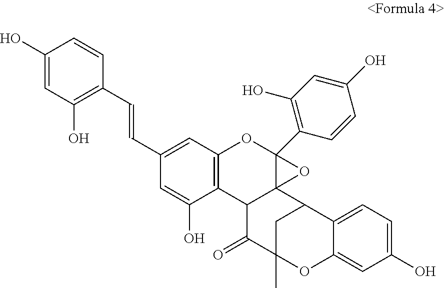 [SMILES: CC12CC(c3ccc(O)cc3O1)C13OC1(c1ccc(O)cc1O)Oc1cc(/C=C/c4ccc(O)cc4O)cc(O)c1C3C2=O]

Oxyresveratrol is shown in Formula 5 and the isolating-preparation from Morus Bark is disclosed in Korean Patent Application No. 2009-112222. Skin-lightening activity is disclosed in the said patent. Also, antioxidative activity (Lorenz P., et al., 2003, Nitric Oxide 9(2):64) and anti-inflammatory activity (Jung K. O., et al., 2003, J Pharm Pharmacol 55(12):1695) is known.

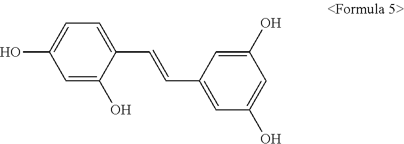 [SMILES: Oc1cc(O)cc(/C=C/c2ccc(O)cc2O)c1]

The activities of the compound of 2′,4′,5,7-tetrahydroxyflavanone as represented in Formula 6 are not known.

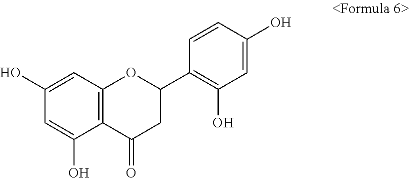 [SMILES: O=C1CC(c2ccc(O)cc2O)Oc2cc(O)cc(O)c21]

The structure of morusignin L as represented in Formula 7 was found in 1993 (Yoshio H., et al., 1993, Heterocycles 36(6) 1359-1366); however the activities are not known.

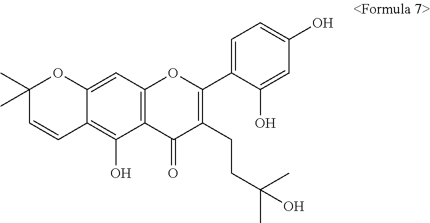 [SMILES: CC(C)(O)CCc1c(-c2ccc(O)cc2O)oc2cc3c(c(O)c2c1=O)C=CC(C)(C)O3]

Dihydromorin as represented in Formula 8 is known to inhibit tyrosinase (Kuniyoshi S., et al., 1998, Plata Medica 64(5) 408-412).

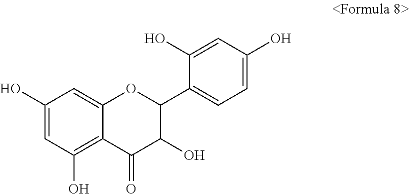 [SMILES: O=C1c2c(O)cc(O)cc2OC(c2ccc(O)cc2O)C1O]

While screening the activity of the compound isolated from Morus Bark, the present inventors have found the compounds of Formula 1 to 8 have effects on inhibiting AGEs which induce diabetic complications from being produced. Thus, the present inventors have completed the present invention after confirming the possibility for the compounds of Formula 1 to 8 to be developed as an AGE inhibitor.

### SUMMARY OF THE INVENTION

Accordingly, the present invention is designed to provide an advanced glycation end product inhibitor which can inhibit an occurrence of diabetic complications by using a compound isolated from Morus Bark.

Moreover, the present invention is designed to provide a health functional food which has a use to improve the diabetic complication inducing circumstances as a secondary symptom of a diabetic patient, by taking a food comprising the said compounds.

The present invention provides AGE inhibitor to inhibit an occurrence of diabetic complications comprising a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin as an active substance.

Advanced glycation end products (AGEs) are the causative substances of inducing diabetic complications. In the kinds of AGEs, there are fluorescence materials such as pentosidine and argpyrimidine, and non-fluorescence materials such as N-carboxymethyl lysine (CML) and N-carboxyethyl lysine (CEL).

Since the experimental method using fluorescence analysis (Monnier, V. M., et al., 1984, Proc. Natl. Acad. Sci. USA 81:583-587) or the method using an antibody specific to AGEs (Horie H., et al., 1997, J. Clin. Invest. 100(12), 2995-3004) are well established, the degree of formulation of AGEs may be identified through the said methods.

By identifying the degree of inhibiting the amount of AGEs with IC₅₀ which measures the amount of AGEs produced in culture medium with microplate reader (Excitation: 360 nm, Emission: 465 nm, fluorescence analysis) of the compounds of mulberrofuran G, mulberrofuran K, kuwanon G, kuwanon Z, oxyresveratrol, 2′,4′,5,7-tetrahydroxyflavanone, morusignin L and dihydromorin and with Western blot analysis using an antibody specific to the typical non-fluorescence substance, CML, it is found that the compounds have an activity inhibiting the production of AGEs which are the causative substances of diabetic complications.

Thus, the compounds of mulberrofuran G, mulberrofuran K, kuwanon G, kuwanon Z, oxyresveratrol, 2′,4′,5,7-tetrahydroxyflavanone, morusignin L and dihydromorin have medicinal use as inhibitors of AGEs, which induce diabetic complications.

Particularly, AGE inhibitor according to the present invention may prevent or treat diabetic nephropathy, diabetic retinopathy or diabetic neuropathy by the inhibitory activity of AGE production since diabetic nephropathy is induced by AGEs (Li, J. H:, et al., 2004, FASEB J. 18, 176-178; Fukami K., et al., 2004, Kidney Int. 66, 2137-2147; Chung, C. K., et al., 2010, J. Am. Soc. Nephrol. 21, 249-260) and diabetic retinopathy and diabetic neuropathy are induced by the interaction with AGE receptors (Barile G. R., et al., 2005, Invest Ophthalmol Vis Sci. 46(8), 2916-2924; Toth C., et al., 2008, Diabetes, 57(4), 1002-1017).

AGE inhibitor according to the present invention could be injected in the various form of perenteral dispensing, however the most preferable route is oral administration. Also, in case of making pharmaceutical preparation, it could be prepared by using diluents or diluting agents such as commonly used filing agent, extending agent, bonding agent, wetting agent, disintegrating agent and surfactant.

The solid preparation for oral administration includes tablets, pills, powders, granules and capsules. Such solid preparations could be prepared by mixing one or more diluting agents, for example microcrystalline cellulose, low substituted hydroxypropylcellulose, colloidal silicon dioxide, calcium silicate, starch, calcium carbonate, sucrose or lactose, and gelatin. Besides the simple diluting agents, a lubricant such as magnesium stearate talc could be used. Moreover, the liquid preparation for oral administration includes suspension, internal solution, emulsion and syrup as well as various diluting agent such as wetting agent, sweetening agent, flavoring agent and preserved agent besides commonly used diluents such as water and liquid paraffin. The preparation for non oral administration includes sterilized aqueous solution, nonaqueous solvent, suspension, emulsion, lyophilized product and suppository. Nonaqueous solvent, propylene glycol as suspension, polyethylene glycol, vegetable oil like olive oil and injectable ester like ethylolate could be used. For suppository, witepsol, macrogol, tween 61, cocoa butter, laurinum and glycerogellatin could be used.

Furthermore, the range of the amount of administration or intake of AGE inhibitor depends on weight, age, sex, health state, diet, administration time, injection method, rate of excretion and severity of disease of patient. For adults, the amount of mulberrofuran G, mulberrofuran K, kuwanon G, kuwanon Z, oxyresveratrol, 2′,4′,5,7-tetrahydroxyflavanone, morusignin L and dihydromorin is 0.1 mg/kg to 1000 mg/kg, taking a dose once or dividing a dose into several times preferably.

The present invention relates to a health functional food comprising a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Morus Bark and comprising a sitologically acceptable food additive.

By taking the food comprising the said compound according to the present invention, the occurrence of diabetic complications as the secondary symptom of a patient with diabetes may be improved. More specifically, the present invention provides the health functional food inhibiting AGE production which could improve the symptom of diabetic complications resulted from AGEs.

According to the present invention, the health functional food inhibiting AGE production comprises a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Morus Bark.

By identifying the degree of inhibiting the amount of AGEs with IC₅₀, which measures the amount of AGEs produced in culture medium with microplate reader (Excitation: 360 nm, Emission: 465 nm, fluorescence analysis) of the compounds of mulberrofuran G, mulberrofuran K, kuwanon G, kuwanon Z, oxyresveratrol, 2′,4′,5,7-tetrahydroxyflavanone, morusignin L and dihydromorin and with Western blot analysis using an antibody specific to the typical non-fluorescence substance, CML, it is found that the compounds have an activity inhibiting the production of AGEs which are the causative substances of diabetic complications.

Thus, the health functional food inhibiting AGE production could he used for improving the symptoms of diabetic nephropathy, diabetic retinopathy or diabetic neuropathy as diabetic complication occurred by AGEs.

The health functional food inhibiting AGE production according to the present invention includes various foods for example drink, gum, tea, vitamin complex, dietery supplement. Also, it could be used in the forms of pill, powder, granule, infusion, tablet, capsule or drink.

The amount of herb extract in food or drink is commonly 0.001 to 10 weight % of total weight of food in case of the health functional food. 0.01 to 1 weight % could be added preferably. In case of the composition of health drink, the ratio of 0.001 to 10 g, preferably 0.01 to 1 g on the basis of 100 ml could be added.

Similar to a common drink, the composition of health drink could comprise various flavoring agents or natural carbohydrate as supplementary ingredient besides comprising the compound isolated from Morus Bark in the indicated ratio as essential substance.

Examples of the said natural carbohydrate are common sugars, such as monosaccharides (e.g. glucose and fructose), disaccharides (e.g. maltose and sucrose), and polysaccharides (e.g. dextrin and cyclodextrin, as well as sugar alcohols such as xylitol, sorbitol and erythritol. As for flavoring agents other than the aforementioned, natural flavoring agents (thaumatin, stevia extracts (for example revaudioside A, glycyrrhizin etc.)) and synthesized flavoring agents (saccharine, aspartame etc) could be used. The ratio of the natural carbohydrate is commonly approximately 1 to 20 g, preferably 5 to 12 g per 100ml of the health functional food of the present invention.

Besides ones mentioned above, the health functional food according to the present invention could comprise various nutritional supplements, vitamins, minerals (electrolytes), synthesized and natural flavoring agents, colorants, filing agents (cheese, chocolate, etc.), pectic acid and salt thereof, alginate and salt thereof, organic acids, protective colloid thickening agents, pH adjustors, immobilizing agents, antioxidants, glycerin, alcohol and carbonizating agents used for soda. Moreover, the health functional food could comprise the fruit flesh for manufacturing natural fruit juice, fruit juice drink and vegetable drink. Such ingredients could be used alone or together. The rate of these additives is not significant however is selected from 0 to approximately 20 per 100 part by weight commonly.

The present invention is used as an AGE inhibitor or a health functional food for inhibiting an occurrence of diabetic complications comprising a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Morus Bark.

Particularly, the present invention is used for preventing or treating diabetic complications such as diabetic nephropathy, diabetic retinopathy or diabetic neuropathy.

### BRIEF DESCRIPTION OF THE DRAWINGS

The drawing is a picture showing the inhibition of AGE production for compounds of Formula 1 to 8.

### DETAILED DESCRIPTION OF THE INVENTION

Hereinafter, exemplary embodiment of the present invention will be described in detail. However, it should be understood that the description proposed herein is just a preferable example for the purpose of illustrations only, not intended to limit the scope of the invention.

### EXAMPLE 1

### Preparation and Analysis of Mulberrofuran G from Morus Bark

1-1: Methanol Extracts from Morus Bark

Dried Morus Bark (3 kg) and methanol (10 L) was undergone repeated reflux extraction three times 4 hours per time. And 150 g of methanol extracts were obtained by vacuum evaporation after filtration.

1-2: Preparation of Organic Solvent Fraction Isolated from Morus Bark Extracts

Hexane extracts (50 g) and ethylacetate extracts (50 g) were obtained by partitioning hexane (3 L, 3 times) and ethylacetate (3 L, 3 times) one by one after suspending methanol extracts (150 g) prepared from 1-1 above in 6 L of water. 25 small fractions (MAE-01˜25) were obtained by applying Phased concentration gradient solvent system consisting of dichloromethane (CH₂Cl₂)-methanol (70%:30%, 50%:50%, 30%:70%, 10%:90%, 0:100%) to ethylacetate extracts in Silica gel column chromatography (silica gel column 500 g).

1-3: Preparation of Active Fraction and Compound from Organic Solvent Fractions

15 small fractions (MA.E-1601˜1615) were obtained by applying Reversed phase silica gel (RP-18) column chromagraphy (methanol:water=1:1→3:1, Methanol, Phased concentration gradient system) to ethylacetate small fraction MA.E-16 prepared in 1-2 above. The fraction MA.E-1607 was undergone Reversed phase column chromatography (RP-18, 2000 ml→2:1 (1000 ml)) and divided into 10 small fractions (160701˜160710). And double MA.E-160709 was undergone by Preparative high performance liquid chromatography (HPLC, Sunfire®C18, 5 μm, 19×150 mm i.d., 50% of acetonitrile, 285 nm, 5 ml/min) and isolate-purified so murberrofuran G (50 mg) of Formula 1 was obtained.

Mulberrofuran G: Amorphous orange-colored powder.

¹H-NMR(500 MHz, CD₃OD): 1.76(3H, s, H-7″), 1.99(1H, dd, J=5.2, 16,9 Hz, H-6″), 2.63(1H, dd, J=11.8, 16.9 Hz, H-6″), 2.92(1H, ddd, J=5.2, 11.8, 11.8 Hz, H-5″), 3.29(2H, H-3″, H-4″), 6.11(1H, dd, J=2.6, 8.6 Hz, H-13″), 629(1H, d, J=2.6 Hz, H-11″), 6.32(1H, d, J=2.3 Hz, H-17″), 6.38(1H, brs, H-2″), 6.43(1H, dd, J=2.3, 8.6 Hz, H-19″), 6.78(1H, d, J=1.4 Hz, H-2′), 6.70(1H, dd, J=2.3, 8.6 Hz, H-5), 6.87(2H, H-7, H-6′), 6.89(1H, H-3), 7.06(1H, d, J=8.6 Hz, H-20″), 7.11(1H, d, J=8.6 Hz, H-14″), 7.30(1H, d, J=8.6 Hz, H-4).

¹³C-NMR(125 MHz, CD₃OD): 22.6(C-7″), 27.5(C-5″), 34.1(C-3″), 35.4(C-6″), 36.3(C-4″), 97.2(C-7), 100.8(C-3), 101.8(C-8″), 102.8(C-17″), 103.2(C-11″), 103.7(C-6′), 104.1(C-2′), 105.7(C-13″), 108.7(C-19″), 111.9(C-5), 112.6(C-4′), 116.0(C-9″), 116.9(C-15″), 120.7(C-4), 121.8(C-3a), 122.0(C-2″), 126.7(C-20″), 129.3(C-14″), 130.2(C-1′), 132.6(C-1″), 152.3(C-16″), 153.6(C-5′), 154.4(C-2), 155.5(C-6), 155.9(C-7a), 156.4(C-18″), 156.6(C-10″), 157.1(C-3′), 158.7(C-12″).

¹H-NMR(500 MHz, Acetone-d₆): 3.34(1H, dd, J=5.5, 12.0 Hz, H-4″)

ESI-MS (negative mode) m/z[M−H]⁻: 561

### EXAMPLE 2

### Preparation and Analysis of Mulberrofuran K from Morus Bark

20 small fractions (MA.E-1401˜1420) were obtained by applying Reversed phase (RP-18) column chromatography (methanol:water=1:1→3:1, Methanol, Phased concentration gradient system) to ethylacetate small fraction, MA.E-14 prepared by the step 1-2 of Example 1. Mulberrofuran K (20 mg) was obtained by applying Reversed phase silica gel column chromatography (RP-18, methanol:water=1:1 (2000 ml→2:1 (1000 ml)) to small fractions, MA.E-1417˜1418 which were divided into 2 small fractions and isolate-purified after applying RP-18.

Mulberrofuran K: Light yellowish crystalline powder.

¹H-NMR(400 MHz, DMSO-d₆): 1.24(3H, s, H-24″), 1.29(3H, s, H-25″), 1.74(3H, s, H-1″), 1.92(1H, dd, J=11.4, 16.2 Hz, H-6″), 2.68(1H, brs, H-6″), 2.77(1H, m, H-5″), 3.18(1H, brs, H-3″), 3.23(1H, dd, J=11.5 Hz, H-4″), 5.67(1H, d, J=9.9 Hz, H-22″), 6.23(1H, brs, H-17″), 6.24(1H, d, J=8 Hz, H-13″), 6.31(1H, brd, J=4.4 Hz, H-2″), 6.41(1H, dd, J=2.4, 8.4 Hz, H-19″), 6.57(1H, d, J=9.9 Hz, H-21″), 6.74(1H, dd, J=2, 8.4 Hz, H-5), 6.83(1H, d, J=1.5 Hz, H-2′), 6.92(3H, 1H×3, H-6′, H-7, H-14″), 7.08(1H, d, J=8.4 Hz, H-20″), 7.11(1H, s, H-3), 7.40(1H, d, J=8.4 Hz, H-4), 9.32, 9.62, 9.79, 9.85(each brs, aromatic OH).

¹³C-NMR(100 MHz, DMSO-d₆): 23.6(C-7″), 26.6(C-24″), 27.1(C-5″), 27.3(C-25″), 33.5(C-3″), 35.4(C-6″), 36.3(C-4″), 75.5(C-23″), 97.4(C-7), 100.6(C-8″), 101.7(C-3), 102.6(C-17″), 103.2(C-6′), 104.2(C-2′), 107.0(C-13″), 109.0(C-19″), 109.8(C-11″), 111.7(C-4′), 112.5(C-5), 115.9(C-15″), 116.8(C-21″), 117.0(C-9″), 120.8(C-3a), 121.2(C-4), 121.4(C-2″), 127.1(C-20″), 127.8(C-14″), 128.6(C-22″), 129.6(C-1′), 132.9(C-1″), 151.2(C-12″), 151.8(C-18″), 153.3(C-2), 153.4(C-3′), 153.8(C-10″), 155.3(C-6), 155.8(C-7a), 156.7(C-16″), 157.1(C-5′).

ESI-MS(negative mode) m/z [M−H]⁻: 627

### EXAMPLE 3

### Preparation and Analysis of Kuwanon-G from Morus Bark

12 small fractions (MA.E-1701˜1712) were obtained by applying Reversed phase silica gel (RP-18) column chromatography (methanol:water=1:1→2:1, Methanol, Phased concentration gradient system) to ethylacetate small fraction, MA.E-17 prepared by the step 1-2 of Example 1. Kuwanon-G (30 mg) shown in Formula 3 was obtained by applying Preparative high performance liquid chromatography (HPLC, Sunfire®C18, 5 μm, 19×150 mm i.d., 60% of acetonitrile, 285 nm, 5 ml/min) to small fraction, MA.E-1710 and isolate-purifying it.

KuwanonG: Amorphous reddish powder.

¹H-NMR(500 MHz, acetone-d₆): 7.34(1H, H-14″), 7.27(1H, H-6′), 6.76(1H, H-20″), 6.65(1H, H-3′), 6.55(1H, H-5′), 6.18(1H, H-17″), 6.06(1H, H-19″), 6.00(1H, H-6), 5.96(1H, H-11″), 5.93(1H, H-13″), 5.20(1H, H-2″), 5.16(1H, H-12), 4.62(1H, H-6″), 4.42(1H, H-1″), 3.70(1H, H-7″), 3.15(1H, H-11), 3.14(1H, H-11), 1.59(3H, H-14), 1.51(3H, H-4″), 1.47(3H, H-15), 1.28(2H, H-5″).

¹³C-NMR(125 MHz, acetone-d₆): 208.6(C-8″), 182.4(C-4), 165.1(C-12″), 164.1(C-10″), 161.5(C-4′), 161.3(C-2′), 160.5(C-7, 9), 156.6(C-5, 18″), 156.3(C-2, 16″), 133.7(C-3″), 132.8(C-14″), 132.0(C-13), 131.4(C-6′), 127.9(C-20″), 123.6(C-2″), 122.1(C-12, 15″), 120.5(C-3), 116.5(C-1′), 115.0(C-9″), 108.0(C-8), 107.4(C-5), 107.0(C-13″), 106.7(C-19″), 104.8(C-10), 103.0(C-3′, 17″), 102.1(C-11″), 97.8(C-6), 47.3(C-7″), 40.4(C-6″), 37.7(C-1″), 29.4(C-5″), 24.9(C-14), 23.6(C-11), 22.2(C-4″), 16.8(C-15).

ESI-MS (positive mode) m/z [M+H]⁺: 693

### EXAMPLE 4

### Preparation and Analysis of Kuwanon-Z from Morus Bark

9 small fractions (MA.E-2301˜2309) were obtained by applying Reversed phase silica gel (RP-18) column chromatography (methanol:water=1:1) to ethylacetate small fraction, MA.E-23 prepared by the step 1-2 of Example 1. Kuwanon-Z (5 mg) shown in Formula 4 was obtained by applying Preparative high performance liquid chromatography (HPLC, Sunfire®C18, 5 μm, 19×150 mm i.d., 45% of acetonitrile, 285 nm, 10 ml/min) to small fraction, MA.E-2305 and isolate-purifying it.

Kuwanon Z: Amorphous orange-colored powder.

¹H-NMR(500 MHz, CD₃OD): 1.68(3H, s, H-7″), 1.84(1H, m, H-6″), 2.71(1H, m, H-6″), 2.73(2H, H-3″, H-5″), 6.06(1H, s, H-17″), 6.17(1H, s, H-6′), 6.27(2H, H-3, H-5), 6.29(1H, d, J=8.3 Hz, H-14″), 6.35(1H, s, H-11″), 6.47(1H, d, J=8.3 Hz, H-19), 6.52(1H, s, H-2′), 6.62(1H, d, J=8.3 Hz, H-13″), 6.75(1H, d, J=16.4 Hz, Hβ), 7.17(1H, d, J=16.4 Hz, Hα), 7.18(1H, s, H-4), 7.26(1H, d, J=8.3 Hz, H-6), 7.44(1H, d, J=8.3 Hz, H-20″).

¹³C-NMR(125 MHz, CD₃OD): 21.1(C-7″), 30.2(C-6″), 37.4(C-3″, C-5″), 74.8(C-1″), 92.1(C-4″), 97.4(C-17″), 98.9(C-2′), 102.2(C-3, C-11″), 106.3(C-6′), 106.8(C-14″), 107.1(C-5), 108.7(C-8″), 108.8(C-9″), 110.1(C-4′), 112.0(C-19″), 113.2(C-15″), 116.5(C-1), 123.2(C-3α), 125.3(C-β), 127.1(C-6), 132.9(C-13″), 140.8(C-1′), 154.4(C-3′), 154.9(C-12″), 155.9(C-2), 157.9(C-4, C-10″), 159.7(C-5′), 168.2(C-18″), 173.0(C-16″), 197.7(C-2″).

ESI-MS negative mode m/z[M−H]⁻: 593, positive mode m/z[M+H]⁺: 595

### EXAMPLE 5

### Preparation and Analysis of Oxyresveratrol from Morus Bark

9 small fractions (MA.E-2301˜2309) were obtained by applying Reversed phase silica gel (RP-18) column chromatography (methanol:water=1:1) to ethylacetate small fraction, MA.E-23 prepared by the step 1-2 of Example 1. Oxyresveratrol (10 mg) shown in Formula 5 was obtained by applying Preparative high performance liquid chromatography (HPLC, Sunfire®C18, 5 μm, 19×150 mm i.d., 17% of acetonitrile, 210 nm, 12 ml/min) to small fraction, MA.E-2301 and isolate-purifying it with approximately 19 minutes of retention time.

Oxyresveratrol: White powder.

¹H-NMR(500 MHz, CD₃OD): 6.12(1H, t, J=2.3 Hz, H-4′), 6.28(2H, m, H-3, H-5), 6.43(2H, d, J=2.3 Hz, H-2′, H-6′), 6.79(1H, d, J=16.3 Hz, H-β), 7.25(1H, d, J=16.3 Hz, H-α), 7.30(1H, d, J=9.2 Hz, H-6).

¹³C-NMR(125 MHz, CD₃OD): 101.0(C-4′), 102.2(C-5), 104.3(C-2′, C-6′), 107.1(C-3), 116.5(C-1), 123.5(C-α), 125.2(C-β), 127.1(C-6), 140.9(C-1′), 156.0(C-2), 157.9(C-4), 158.2(C-3′, C-5′).

ESI-MS (negative mode) m/z [M−H]⁻: 243.

### EXAMPLE 6

### Preparation and Analysis of 2′,4′,5,7-Tetrahydroxyflavanone from Morus Bark

20 small actions (MA.E-1401˜1420) were obtained by applying Reversed phase silica gel (RP-18) column chromatography (methanol:water=1:1→3:1, Methanol, Phased concentration gradient system) to ethylacetate small fraction, MA.E-14 prepared by the step 1-2 of Example 1. 2′,4′,5,7-tetrahydroxyflavanone (5 mg) shown in Formula 6 was obtained by applying Reversed phase column chromatography (RP-18, methanol:water=1:1) to small fraction. MA.E-1403, wherein MA.E-1403 was divided into 6 small fractions (140301˜140306) and MA.E-140303 among them was isolate-purified after applying RP-18.

2′,4′,5,7-tetrahydroxyflavanone: Yellowish powder.

¹H-NMR(500 MHz, CD₃OD): 2.69(1H, dd, J=3.2, 17.3 Hz, H-3), 3.04(1H, dd, J=13.2, 17.3 Hz, H-3), 5.58(1H, dd, J=3.2, 13.2 Hz, H-2), 5.86(1H, d, J=2.0 Hz, H-6), 5.89(1H, d, J=2.0 Hz, H-8), 6.30(1H, d, J=2.3 Hz, H-3′), 6.32(1H, dd, J=2.3, 8.1 Hz, H-5′), 7.21(1H, d, J=8.1 Hz, H-6′).

¹³C-NMR(125 MHz, CD₃OD): 41.8(C-3), 74.6(C-2), 94.8(C-8), 95.6(C-6), 102.0(C-9), 102.1(C-3′), 106.5(C-5′), 116.6(C-1′), 127.5(C-6′), 155.5(C-2′), 158.4(C-4′), 164.1(C-5), 164.2(C-10), 167.0(C-7), 197.2(C-4).

ESI-MS (negative mode) m/z [M−H]⁻: 287

### EXAMPLE 7

### Preparation and Analysis of Morusignin L from Morus Bark

13 small fractions (MA.E-2201˜2213) were obtained by applying Reversed phase silica gel (RP-18) column chromatography (methanol:water=1:1→3:1, Methanol, Phased concentration gradient system) to ethylacetate small fraction, MA.E-22 prepared by the step 1-2 of Example 1. Morusignin L (5 mg) shown in Formula 7 which yellow spots turn up when color developing with dilute sulfuric acid agent and have Rf value of 0.15 in Thin-Layer Chromatography (TLC) was obtained by applying MCI gel (Supelco company) ion-exchange chromatography to fraction, MA.E-2212 and isolate-purifying it.

Morusignin L: Yellowish powder.

¹H-NMR(500 MHz, acetone-d₆): 1.05(6H, H-4″, H-5″), 1.42(6H, 2CH₃), 1.60(2H, H-2″), 2.48(2H, H-1″), 5.62(1H, H-3), 6.12(1H, H-10), 6.50(1H, H-5′), 6.54(1H, H-3′), 6.57(1H, H-4), 7.26(1H, H-6′).

¹³C-NMR(125 MHz, acetone-d₆): 20.3(C-1″), 27.5(2CH₃), 28.5(C-4″, C-5″), 42.2(C-2″), 69.3(C-3′), 77.9(C-2), 98.9(C-10), 100.7(C-4a), 103.2(C-3′), 104.8(C-5a), 107.4(C-5′), 112.0(C-1′), 114.6(C-4), 122.1(C-6), 127.2(C-3), 131.4(C-6′), 152.5(C-9), 156.4(C-2′), 159.1(C-1a), 160.6(C-4′), 161.5(C-7), 162.0(C-8a), 182.7(C-5).

ESI-MS (positive mode) m/z [M+H]⁻: 439

### EXAMPLE 8

### Preparation and Analysis of Dihydromorin from Morus Bark

9 small fractions (MA.E-2301˜2309) were obtained by applying Reversed phase silica gel (RP-18) column chromatography (methanol:water=1:1) to ethylacetate small fraction, MA.E-23 prepared by the step 1-2 of Example 1. Dihydromorin (10 mg) shown in Formula 8 was obtained by applying Preparative high performance liquid chromatography (HPLC, Sunfire®C18, 5 μm, 19×150 mm i.d., 17% of acetonitrile, 210 nm, 12 ml/mm) to small fraction, MA.E-2301 and isolate-purifying it with approximately 17 minutes of retention time.

Dihydromorin: Yellowish powder.

¹H-NMR(500 MHz, CD₃OD): 4.74(1H, J=11.5 Hz, H-3), 5.36(1H, J=11.5 Hz, H-2), 5.81(1H, J=2.3 Hz, H-6), 5.85(1H, J=2.3 Hz, H-8), 6.32(1H, H-3′), 6.33(1H, H-5′), 7.20(1H, H-6′).

¹³C-NMR(125 MHz, CD₃OD): 71.7(C-3), 78.6(C-2), 95.5(C-6), 96.3(C-8), 101.1(C-4a), 102.3(C-5′), 106.5(C-3′), 114.3(C-1′), 129.5(C-6′), 157.3(C-2′), 158.1(C-4′), 163.6(C-5), 164.0(C-8a), 169.3(C-7), 197.0(C-4).

ESI-MS (negative mode) m/z [M−H]⁻: 303

### EXPERIMENTAL EXAMPLE 1

### Assay of Inhibition Effect on AGE Production

After preparing 10 mg/ml bovine serum albumin (BSA) dissolved in 50 mM of phosphate buffer (pH 7.4), AGE production was induced by culturing the mixture of 0.2 M of fructose and glucose at 37□ for 7 days. The compounds extracted from Morus Bark and prepared from Example 1 to 8 was handled in the concentration of 0.1 μg/Ml respectively. (All the compounds were dissolved in 100% of ethanol.) Pyridoxamine known for AGE inhibitor was used as a positive control group. AGE production was induced by using only fructose and glucose to BSA culturing at 37□ for 7 days. It was handled in the concentration of 0.1 μg/Ml to 1000 μg/Ml.

After 7 days, the amount of AGE produced in culture medium was measured by using microplate reader (Excitation: 360 nm, Emission: 465 nm, fluorescence analysis). As a result, IC₅₀ value was calculated by using Sigma plot program as shown in Table 1.

The rate of inhibiting the production was counted by the following numerical formula. All the experiments were demonstrated 2 times per sample. Also, the average and standard deviation of IC₅₀ value were calculated from at least 3 independent experiments.

The rate of inhibiting the production (%)=100−(Fluorescence intensity of Experimental group−Fluorescence intensity of Reference)/(Fluorescence intensity of Control group−Fluorescence intensity of Control-Reference)×100

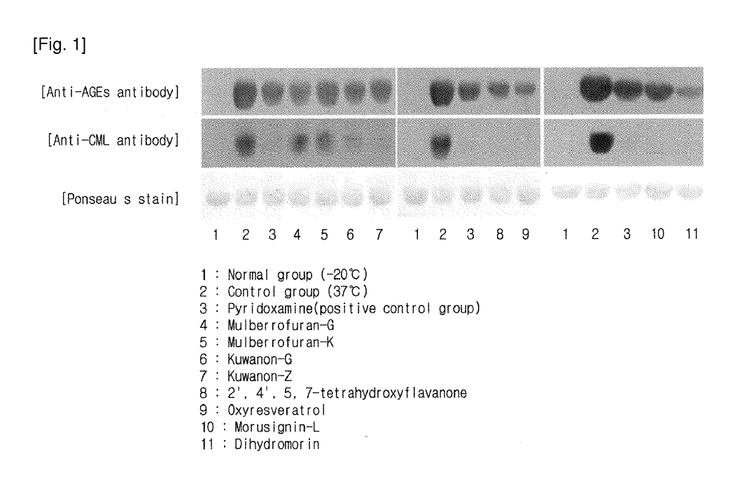

Moreover, false positive which could be found in fluorescence analysis was confirmed through western blot analysis using antibodies specific to non-fluorescence substance, CML which is representative of antibodies specific to AGE, or the kinds of AGE as shown in FIG. 1.

TABLE 1

| | | IC₅₀ value | IC₅₀ value |
|---|---|---|---|
| | Active substance | (μg/Ml) | (μM) |
| Example. 1 | Mulberrofuran G | 44.7 ± 10.9 | 79.5 ± 19.4 |
| Example. 2 | Mulberrofuran K | 51.6 ± 4.5 | 82.2 ± 7.2 |
| Example. 3 | Kuwanon G | 57.2 ± 4.5 | 82.7 ± 6.5 |
| Example. 4 | Kuwanon L | 27.4 ± 7.0 | 46.1 ± 11.8 |
| Example. 5 | Oxyresveratrol | 4.7 ± 1.0 | 19.3 ± 4.1 |
| Example. 6 | 2′,4′,5′,7- | 27.7 ± 2.9 | 96.2 ± 10.1 |
| | tetrahydroxyflavanone | | |
| Example. 7 | Morusignin L | 70.1 ± 7.3 | 160.1 ± 16.7 |
| Example. 8 | Dihydromorin | 24.9 ± 1.0 | 81.9 ± 3.3 |
| Positive control | Pyridoxamine | 125.7 ± 35.8 | 747.3 ± 212.8 |
| grp. | | | |

As shown in Table 1, the compounds prepared in Example 1 to 8 according to the present invention have the inhibitory effect on AGE production from 3.7 times to 38.7 on mol concentration (μM) compared to pyridoxamine as a positive control group.

Furthermore, as shown in the drawing, the compounds prepared in Example 1 to 8 according to the present invention have the inhibitory effect on AGE production more than pyridoxamine in the single concentration of 50 μg/Ml through western blot analysis using CML specific antibody and antibody specific to AGE.

Therefore, the compounds prepared in Examples 1 to 8 according to the present invention are useful for AGE inhibitor and health functional food which could prevent and treat diabetic complications such as diabetic nephropathy, diabetic retinopathy and diabetic neuropathy induced by production of AGE of patients having diabetes.

1. An advanced glycation end product (AGE) inhibitor for inhibiting an occurrence of diabetic complications, comprising a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Moms Bark as an active ingredient.

2. The advanced glycation end product (AGE) inhibitor according to claim 1, wherein a diabetic complication is selected from the group consisting of diabetic nephropathy, diabetic retinopathy and diabetic neuropathy.

3. A health functional food for inhibiting an occurrence of diabetic complications, comprising a compound selected from the group consisting of mulberrofuran G, mulberrofuran K, kuwanon G, kuwanon Z, oxyresveratrol, 2′,4′,5,7-tetrahydroxyflavanone, morusignin L and dihydromorin isolated from Morus Bark
